(19) United States
(12) Patent Application Publication
(10) Pub. No.: US 2014/0039204 A1
(43) Pub. Date: Feb. 6, 2014

(54) 7'-PHLOROECKOL COMPOUND FOR HAIR PROLIFERATION AND A COSMETIC COMPOSITION CONTAINING SAME

(71) Applicants: Se-Kwon Kim, Busan (KR); Soon-Sun Bak, Busan (KR)

(72) Inventors: Se-Kwon Kim, Busan (KR); Soon-Sun Bak, Busan (KR)

(73) Assignee: PUKYONG NATIONAL UNIVERSITY INDUSTRY-UNIVERSITY COOPERATION FOUNDATION, Busan (KR)

(21) Appl. No.: 13/978,185

(22) PCT Filed: Mar. 24, 2011
(86) PCT No.: PCT/KR2011/002033

§ 371 Date: Sep. 18, 2013

(30) Foreign Application Priority Data
Jan. 3, 2011 (KR) 10-2011-0000141

(51) Int. Cl. A61K 8/49; A61Q 7/00

(52) U.S. Cl. CPC A61K 8/498; A61Q 7/00
USPC 549/359

(57) ABSTRACT

The effects on human hair growth of 7′-phloroeckol (1-(3′,5′-dihydroxyphenoxy)-7-(2″,4″,6-trihydroxyphenoxy)-2,4,9-trihydroxydibenzo-1,4-dioxin) isolated from E. cava (Ecklonia cava) have been studied in cell systems. The cell-proliferation and anti-apoptotic effects on human human hair follicle dermal papilla cells (HFDPC) and human keratinocytes (human epidermal keratinocytes: HaCaT) were investigated using the 3-(4,5-dimethylthiazol-2-yl)-2,5-diphenyl tetrazolium bromide (MTT) assay and western blot. The 7′-phloroeckol significantly increased HFDPC and HaCaT proliferation. With HFDPC and HaCaT, the amount of PI3K and phosphorylated AKT protein expression increased in a way which was dependent on the extent of 7′-phloroeckol treatment. Also, the levels of MEK½, phosphorylated ERK and c-Fos proteins, which are associated with cell proliferation, increased in a way which was dependent on the extent of 7′-phloroeckol treatment. Meanwhile, apoptosis was inhibited as there was an increase in the Bcl-2/Bax ratio which is associated with apoptosis, and there was a significant reduction in p53 and also caspase-3 and caspase-9. These results indicate that 7′-phloroeckol can promote human hair growth by inhibiting apoptosis and stimulating HFDPC and HaCaT proliferation.

### TECHNICAL FIELD

The present invention relates to 7′-phloroeckol compound for hair proliferation and a composition containing the same. In particular, the invention relates to 7′-phloroeckol compound which could grow human hair affecting Hair Follicle Dermal Papilla Cells (HFDPC) and human keratinocyte (HaCaT) cell line, and a composition containing the same.

### BACKGROUND ART

Human hair is crucial because not only for protecting the skin and scalp but also social and sexual communication. Hair is composed of ketatin protein and grows from follicles found in the dermis. Hair follows a specific growth cycle with anagen (growing phase), catagen (involution phase or transition phase) and telogen (resting phase) (Stenn and Paul, 2001). Scalp follicle is composed of follicle dermal papilla cells, keratinocyte cell line, inner and outer root sheath cells, and melanocyte. Human Follicle Dermal Papilla Cells (HFDPC) are one of the specific fibroblasts which take part in the morphogenesis of follicles (Jahoda et al., 1984, Ferraris et al., 1997). HFDPC are related to hair proliferation with normal and onset conditions (Inui et al., 2003), thus changes in cytokine levels and transcription factor of HFDPC become to be critical (Kwon et al., 2008). Keratin protein is composed from human keratinocyte cell line and main tissue of hair (McGrath et al., 2004). Recently, using various molecular analysis system, cell growth (proliferation), cell lysis and apoptosis-relating factor during anagen phase have been determined. ERK (Extracellular signal-regulated kinases) and AKT (a serine/threonine protein kinase) pathways were shown to related to cell growth of scalp dermal papilla cells (Han et al., 2004, Yoo et al., 2007 and Kwon et al., 2008). ERK (Extracellular signal-regulated kinases) was well known to play certain role in the mitogenesis or cell growth, AKT (a serine/threonine protein kinase) was reported to have an important role in mediating survival signaling. AKT also is activated by growth factor and cytokine and induced through a pathway involving PI3K (Phosphatidylinositol 3-kinases).

Also, p53, Bcl-2, Bax and caspase-3, -9 were known to be affected by apoptosis in scalp follicle cell and keratonocyte cell line (Tsuji et a/.,2003, Winiarska et al., 2006, Kwon et al.,2007). Of these, Bcl-2 family protein is composed of functional proteins in anti-apoptosis or pro-apoptosis and acts as a gatekeeper of the apoptotic processes.

Several preliminary arts related with stimulation of hair growth have been reported, for example, KR 10-2008-0096649 “Composition for treating hair or scalp to prevent hair loss and promote growing hair” describes a composition for treating scalp comprising zinc pyrithione, panthenone and salicylic acid as active ingredient, KR 10-1992-0702114 “A method for stimulating hair growth and treating skin disease, and its products” describes a method stimulated hair growth by applying the composition comprising anol, anethole locally to the affected areas of patient. KR 10-1991-0016248 “ Hair growing composition” describes a composition for growing hair comprising the extracts of Cornus officinalis S, et Z (Cornaceae), Eclipta prostrata (Compositae), Mulberry alba (Moraceae) as active ingredient.

Thus, while various species of herbs for stimulation of hair growth were reported, the stimulant derived from natural marine organism rarely was known, KR 10-2008-18124 “Hair growth stimulator based on ecklonia cava extract, preparation method thereof, and cosmetic composition containing the same” simply describes a composition containing the extracts of several seaweed.

Natural marine organism has lots of useful physiological active compounds, recently many researchers have studied the development and application of said natural marine organism. Sea Algae among natural marine organism can be classified on the basis of pigments, as brown alga (Phaeophyceae), red alga (Rhodophyceae) and green alga (Chlorophyceae).

It was reported that Ecklonia cava (E. cava) is one of the brown alga species found in the ocean off Korea and Japan and has an effect of anticoagulant, antioxidant, antitumor agent and matrix metalloproteinase (MMP) inhibition. In the prior art, it has reported that 7-phloroeckol, one of the physiological active compounds isolated from Ecklonia cava has an inhibitory effect on adipogenesis, MMP and melanogenesis.

We verified the effects on proliferation of HFDPC and HaCaT and anti-apoptotic activities mediated by 7′-phloroeckol and investigated through its action mechanism, resulted in finding the use of 7′-phloroeckol as growth factor for hair.

### DETAILED DESCRIPTION OF THE INVENTION

### Technical Problem

The objective of the present invention is to induce the proliferation of HFDPC and HaCaT which take part in follicle morphology of hair growth cycle and also to determine a compound that is easy to supply materials and rarely harmful to human body, for improving the proliferation of human hair that play major roles in protecting the skin and scalp and social and sexual communication, and preventing the hair from falling out.

### Technical Solution

The present invention provides with following the formula 1 of 7′-phloroeckol which has an effect on hair proliferation:

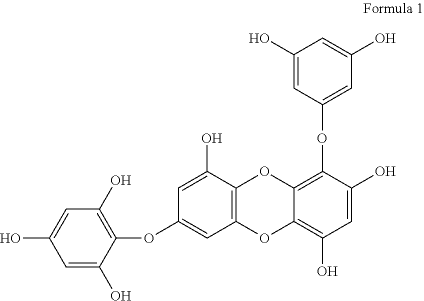 [SMILES: Oc1cc(O)cc(Oc2c(O)cc(O)c3c2Oc2c(O)cc(Oc4c(O)cc(O)cc4O)cc2O3)c1]

### Effect of the Invention

And the invention provides with the above compound characterized by extracting from Ecklonia cava.

Also the invention provides with a composition for hair proliferation characterized by containing 7′-phloroeckol as active ingredient.

### BRIEF DESCRIPTION OF DRAWINGS

FIG. 1 is a graph showing the effect of 7′-phloroeckol on the proliferation of HFDPC by using MTT assay.

FIG. 2 is a graph showing the effect of 7′-phloroeckol on the proliferation of HaCaT by using MTT assay.

FIG. 3 is a graph showing the effect of 7′-phloroeckol on the ERK (Extracellular signal-regulated kinases) pathway in cultured HFDPC (a) and HaCaT (b).

FIG. 4 is a graph showing the effect of 7′-phloroeckol on the AKT pathway in cultured HFDPC (a) and HaCaT (b).

FIG. 5 is a graph showing the effect of 7′-phloroeckol on apoptosis-related factor in HFDPC (a) and HaCaT (b).

FIG. 6 is a graph showing the effect of 7′-phloroeckol on the caspase-3 and caspase-9 in HFDPC (a) and HaCaT (b).

### DESCRIPTION OF EMBODIMENTS

7′-Phloroeckol used in present invention is expressed as following formula, and its IUPAC name is 1-(3′,5′-dihydroxyphenoxy)-7-(2″,4″,6-trihydroxyphenoxy)-2,4,9-trihydroxydibenzo-1,4-dioxin.

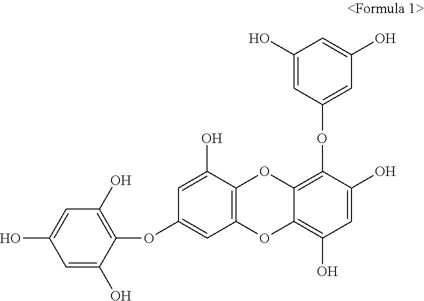 [SMILES: Oc1cc(O)cc(Oc2c(O)cc(O)c3c2Oc2c(O)cc(Oc4c(O)cc(O)cc4O)cc2O3)c1]

7′-Phloroeckol has been known to be one of polyphenolic composites found in E. cava, which has various biological effect, for example antitumor, anti-HIV-1, anti-inflammatory, anti-allergic and anti-adipocyte differentiation.

The invention is not limited to the method of preparation or availability of 7′-phloroeckol, which preferably is extracted from E. cava. More preferably, freeze-dried E. cava is extracted by using ethanol, then the extract is divided by using n-hexane, dichloromethane, EtOAc, n-butanol, H₂O, then EtOac fraction layer, the highest activity of these is divided to isolate active materials from using column chromatography and Sephadex LX-20 column chromatography. 7′-Phloroeckol, active materials, of structure was analyzed by using the known method.

7′-Phloroeckol according to the invention may be included in a cosmetic composition for hair proliferation. Preferably, 7′-phloroeckol is included in the cosmetic composition in a range of effective concentration from 0 to 1.0 μM.

The cosmetic composition may be provided in a variety of products in fields such as, but not limited to, creams, ointments, solutions, emulsion, suspensions foundation and sprays foundation. The cosmetic composition may be provided in the conventional manner that provide such forms in the art by comprising the composition of the invention.

Cosmetic composition may be applied with a conventional dose to scalp in conventional manner in the art.

The following examples further describe and demonstrate embodiments within the scope of the present invention. The examples are given solely for the purpose of illustration and are not to be construed as limitations of the invention.

### Materials Preparation

7′-Phloroeckol was isolated from E. cava according to the prior art. That is, freeze-dried E. cava is extracted by using ethanol, then the extract is divided by using n-hexane, dichloromethane, EtOAc, n-butanol, H₂ O, then EtOac fraction layer, the highest activity of these is divided to isolate active materials from using column chromatography and Sephadex LX-20 column chromatography. 7′-Phloroeckol, active materials, of structure was analyzed by using the known method.

Antibody binding phosphor-ERK-½ (Thr202/Tyr204), whole ERK-½, p53 and c-Fos was purchased from Cell Signaling Technology, Inc. (Beverly, Mass., USA).

Antibody against Bcl-2, caspase-3 and caspase-9 was purchased from BD Biosciences Pharmingen (San Jose, Calif., USA), phosphor-Akt (Ser473), whole Akt, PI3K, MEK-½, Bax and β-actin antibodies were purchased from Santa Cruz Biotech Inc. (Santa Cruz, Calif., USA).

### Cell Culture

Human Hair Follicle Dermal Papilla Cells (HFDPC) were purchased from Promo Cell (Heidelberg, Germany). Cells were cultured in ready-to-use HFDPC medium, which is composed of Basal Medium, Fetal Calf Serum (FCS), Supplement Kit (Promo Cell, Heidelberg, Germany) including basic FGF and insulin. Kit including HEPES Buffer Saline Solution (HBSS), trypsin/EDTA solution and Trypsin Neutralizing Solution (Promo Cell, Heidelberg, Germany) was used for subculture. Subcultured 4^{th} to 6^{th} TFDPC was used for experiments. HaCaT (human epidermal keratinocyte) was cultured in Dulbecco's modified Eagle's medium (DMEM, Gibco BRL,

### Statistical Analyses

Data were expressed as the mean±SEM. The data of each group were assessed by one-way ANOVA with Duncan's multiple range test using the statistical software package SAS v9.1 (SAS Institute Inc., Cary, N.C., USA). P<0.05 was considered to indicate a statistically significant difference.

3-[4,5-dimethylthiazol-2yl]-2,5 diphenyl tetrazolium bromide (MTT) Assay

Cell proliferation of HFDPC and HaCaT were measured by using MTT method. After cells were seeded in 96-well with serum-free medium, the cells were incubated for 1 day (1.0×10⁴ cells/well). Then, we replaced with new medium and cells were treated with 0.01 μM, 0.05 μM, 0.1 μμM, 0.5 μM and 1 μM samples, then incubated for 5 days. MTT solution (5 mg/ml) was added, the cells were cultivated in 37° C. incubator for 3 hours. Then, the medium were removed and the formazan crystal were dissolved with Dimethyl sulfoxide (DMSO), then the absorbance of the solution were measured.

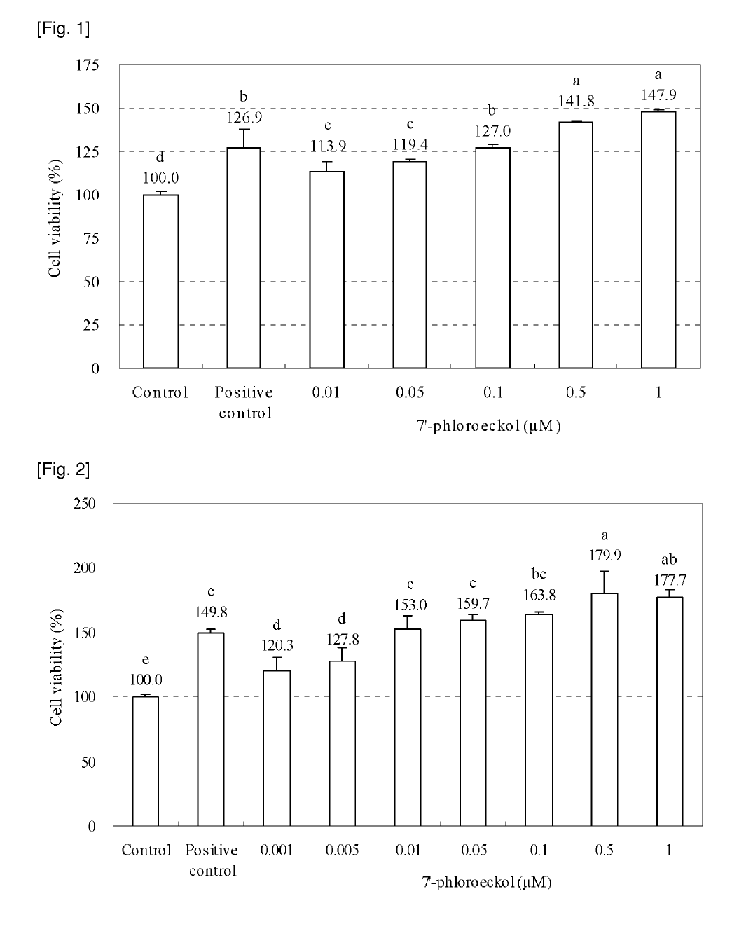

As described in the FIG. 1 and FIG. 2, data were expressed as the percentage on the control that doesn't contain the treated samples. These data is mean±SEM. Different letters as stated on the graph indicate a significant difference in Duncan's multiple range test (p<0.05).

7′-Phloroeckol in HFDPC increased the cell proliferation in dose-dependent manner (p>0.05). 1μM 7′-phloroeckol showed significantly higher cell viability than 1 μM Minoxidil (well known as hair growth solution) used as positive control. Cell proliferation mediated by 0.5 to 1μM 7′-phloroeckol weren't significantly different (FIG. 1).

7′-Phloroeckol in HaCaT also increased the cell proliferation in dose-dependent manner (p>0.05). 0.1 μM 7′-phloroeckol showed higher cell viability than 0.1 μM Minoxidil used as positive control (FIG. 2). 1 μM Minoxidil decreased cell viability than compared to 0.1 μM Minoxidil (these data were not illustrated).

### Western Blot Assay

Western blot assay was performed as standard procedure to study the action mechanism on HFDPC and HaCaT proliferation mediated by 7′-phloroeckol. That is, cells were seeded with serum free medium in 6-well, then cultured for 24 hours (1.0×10⁶ cells/well). Then, the cells were replaced with new medium and treated with the samples in the different concentrations for 1 hour to 24 hours. The cells were lysed in RIPA lysis buffer at 4° C. for 10 minutes. 10 μg cell lysate were divided by 10% SDS-polyacrylamide gel electrophoresis, transferred to polyvinylidenefluoride membrane filter and the membranes were blocked with 5% skim milk, hybridized with primary antibodies (1:10,000 dilution). Following the incubation with secondary horseradish peroxidase-conjugated antibodies at room temperature, immunoreactive protein was detected by using chemiluminescence ECL assay kit according to the manufacturer's instructions. Western blot band was visualized by using LAS3000® Luminescent image analyzer (Fujifilm Life Science, Tokyo, Japan).

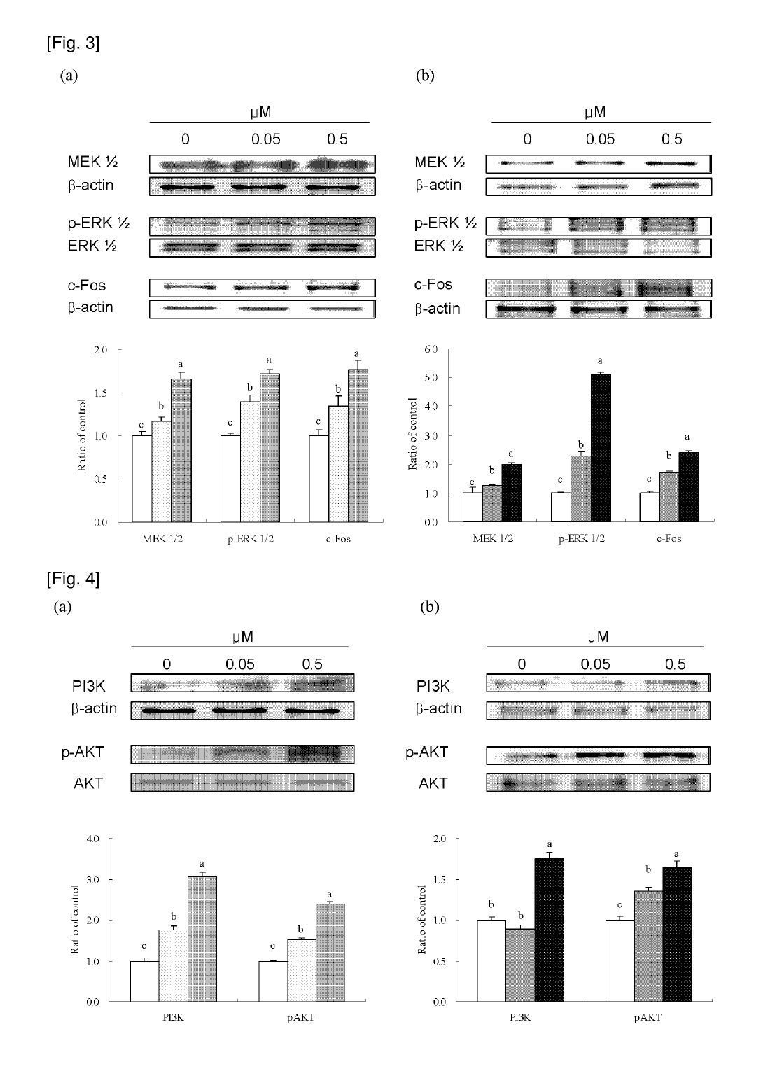

Cells were treated with 7′-phloroeckol for 1 hour to 24 hours to examine the expression of protein by using western blot assay. Graph showing the result of western blot in HFDPC were illustrated in the FIGS. 3 to 6(a) and graphs showing the result of western blot in HaCaT were illustrated in the FIGS. 3 to 6(b). While HFDPC and HaCaT were treated with 7′-phloroeckol for 1 hour in the experiment on the pathway of ERK and AKT relating cell proliferation, HFDPC and HaCaT were treated with 7′-phloroeckol for 24 hours in the experiment on Bcl-2, Bax, p53, caspase-9 and caspase-3 relating apoptosis, then the protein expression were observed.

Data were expressed as a ratio of control group that doesn't contain the treated samples, the values are means±SEM, different letters written on the graph indicate a significant defference in Duncan's multiple range test (p<0.05).

Activation of ERK Pathway Mediated 7′-phloroeckol in HFDPC and HaCaT

HFDPC and HaCaT were treated with 7′-phloroeckol and cultured for 1 hour to study activation of 7′-phloroeckol on ERK pathway related to cell proliferation (FIG. 3). 0.05 and 0.5 μM 7′-phloroeckol increased the expression of MEK ½ protein 1.17-fold and 1.65-fold (p<0.05) than control group. Concentration level of phospho-ERK (p-ERK) in HFDPC that was treated with 0.05 and 0.5 μM 7′-phloroeckol were increased 1.39-fold and 1.71-fold than control group, respectively. But there was no significant difference between total ERK levels treated with sample and without sample. Also, 7′-phloroeckol as described above increased the expression of c-Fos protein (p<0.05). Similar to HaCaT, the expression of MEK ½, p-ERK and c-Fos proteins that were treated with 7′-phloroekol increased in dose-dependent manner. These results suggest that 7′-phloroeckol may affect ERK pathway in HFDPC and HaCaT, resulted in the activation.

Activation of AKT Pathway Mediated 7′-phloroeckol in HFDPC and HaCaT

After HFDPC and HaCaT were treated with 7′-phloroeckol and cultured for 1 hour to study the activation of 7′-phloroeckol on AKT pathway related cell proliferation (FIG. 4). 0.05 and 0.5μM 7′-phloroeckol increased PI3K levels 1.76-fold and 3.07-fold than control group. The expression of phospho AKT (p-AKT) protein in HFDPC that was treated with above level of 7′-phloroeckol was increased 1.53-fold and 2.40-fold compared to the control group. But, there was no difference in whole AKT levels between treated with samples and without samples. Also, 0.5 μM 7′-phloroekol induced 1.75-fold and 1.65-fold levels of PI3K and pAKT in HaCaT. These results suggest that 7′-phloroekol may activate AKT pathway in HFDPC and HaCaT.

Increase of Bcl-2 and Decreases of Bax and p53 Mediated 7′-phloroeckol in HFDPC and HaCaT

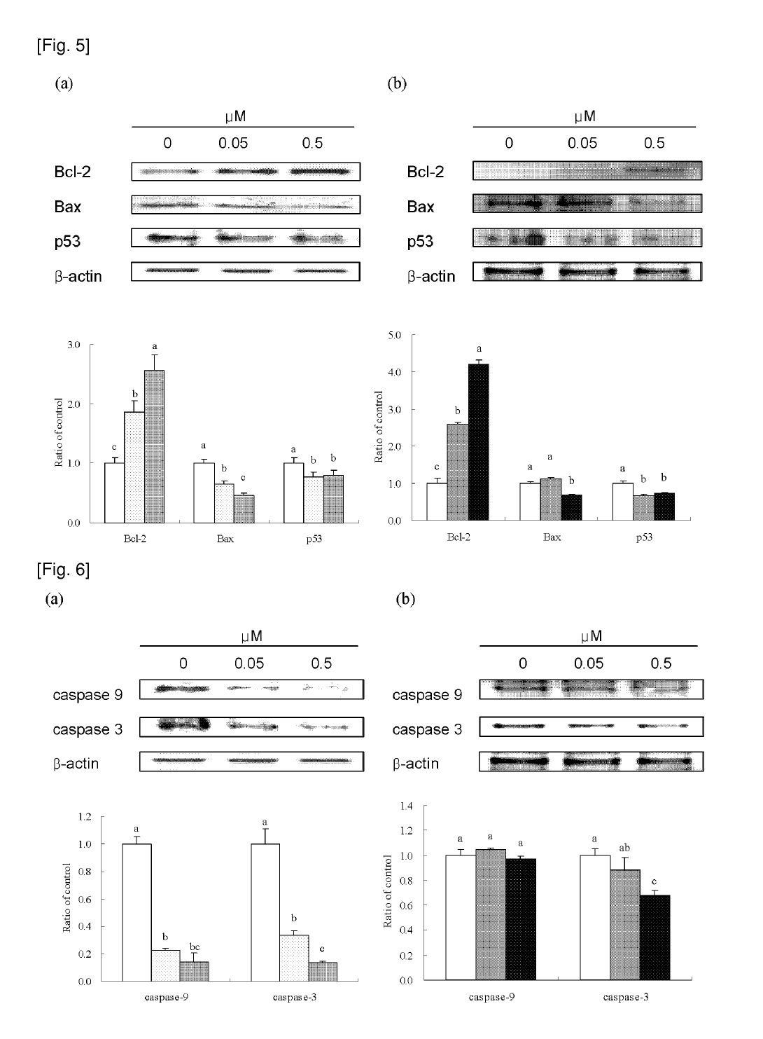

To study the apoptotic effect by 7′-phloroeckol, HFDPC and HaCaT were treated with 7′-phloroeckol, then cultured for 24 hours (FIG. 5).

7′-Phloroeckol significantly increased the expression level of Bcl-2 protein in dose-dependent manner. The expression levels of Bcl-2 and p53 were decreased in HFDPC that was treated with 7′-phloroeckol (P<0.05). Also, the expression of Bcl-2 protein was increased 2.60-fold and 4.21-fold in 0.05 and 0.5 μM HaCaT, respectively, compared to the control group. On the other hand, 0.5 μM 7′-phloroeckol decreased 0.67-fold and 0.72-fold the levels of Bax and p53 in HaCaT (p<0.05). These results suggest that 7′-phloroeckol may have anti-apoptotic effect against HFDPC and HaCaT.

Decreases of caspase-9 and caspase-3 Mediated 7′-phloroeckol in HFDPC and HaCaT

To study the expression of caspase-9 and caspase-3 proteins by western blot assay, HFDPC and HaCaT were treated with 7′-phloroekol and cultured for 24 hours (FIG. 6).The expression of caspase-3 and caspase-9 proteins were decreased 0.14-fold and 0.13-fold in HFDPC that was treated with 0.5μM 7′-phloroeckol (p<0.05) compared to the control group. The expression of caspase-3 protein was decreased 0.88-fold and 0.68-fold in HaCaT that was treated with 7′-phloroeckol in 0.05 and 0.5μM concentration levels, respectively compared to the control group. Thus these results indicate that 7′-phloroeckol may affect kinase, such as caspase-9 and caspase-3 in HFDPC and HaCaT, resulted in the inhibition of the activities.

In the results, 7′-phloroeckol in the level of greater than 0 and less than 1μM significantly induced the proliferation of HFDPC and HaCaT. According to the results, 0.5 and 1.0μM 7′-phloroekol increased the growth rate of HFDPC and HaCaT and affected the expression changes of ERK, ATK and Bcl-2 family protein. That is, 7′-phloroeckol was shown to increase the phosphorylation of ERK and AKT significantly, HFDPC and HaCaT survival were considered to prolong by the activation of cell proliferation-related factors ERK and ACK mediated by 7′-phloroeckol. HFDPC is only Bcl-2 positive area in the hair cycle, in particular Bcl-2 action in HFDPC inhibits cell death in growing phase (anagen) and seems to support the cell proliferation. Lindler et al had reported not to observe Bax coloration in HFDPC for all experimented hair cycle, but recent study showed to observe the expression of Bax. We observed the expression of Bax protein in HFDPC and HaCaT, 7′-phloroekol increased the expression of antiapoptotic molecules Bcl-2, but decreased the expression of proapoptotic molecule Bax. 7′-Phloroekol was shown to inhibit apoptosis by suppressing the expression of caspase-9 and caspase-3 related to apoptosis in HFDPC and HaCaT. These results indicate that 7′-phloroekol induce cell survival by controlling Bcl-2 and Bax proportions in HFDPC and HaCaT and inhibiting caspase-9 and caspase-3.

Consequently, 7′-phloroeckol was first shown to affect the expression of ERK, Akt, Bcl-2, Bax, caspase-9 and caspase-3 in HFDPC and HaCaT. The invention also demonstrated that 7′-phloroeckol induced the cell proliferation, but inhibited cell death by increasing the viabilities of HFDPC and HaCaT and a ratio of Bcl-2/Bax and decreasing the expression of kinase such as caspase-9 and caspase-3 through ERK and AKT signaling pathway. 7′-Phloroeckol through such effects of proliferation and antiapoptosis may stimulate human hair growth, could extend anagen phase, also have a proliferative effect on other cell type of follicle other than HFDPC and HaCaT.

What is claimed is:

1. A 7′-phloroeckol compound for human hair growth of formula 1

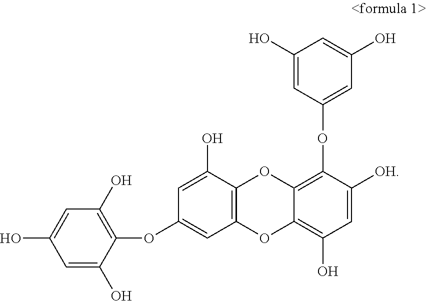 [SMILES: Oc1cc(O)cc(Oc2c(O)cc(O)c3c2Oc2c(O)cc(Oc4c(O)cc(O)cc4O)cc2O3)c1]

2. The compound of claim 1, wherein the compound is extracted from E. cava.

3. A composition for human hair growth comprising 7′-phloroeckol compound of claim 1 as an active ingredient.
